(19) United States
(12) Patent Application Publication
(10) Pub. No.: US 2008/0095751 A1
(43) Pub. Date: Apr. 24, 2008

(54) Atherosclerosis

(75) Inventors: Chunming Dong, Durham, NC (US); Pascal J. Goldschmidt, Miami, FL (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/790,809

(22) Filed: Apr. 27, 2007

Related U.S. Application Data
(60) Provisional application No. 60/795,203, filed on Apr. 27, 2006.
(60) Provisional application No. 60/854,695, filed on Oct. 27, 2006.

(51) Int. Cl. A61K 35/12; A61P 9/10; C12N 5/06

(52) U.S. Cl. 424/93.21; 424/93.7; 435/325; 435/366; 435/371; 435/372

(57) ABSTRACT

The present invention relates, in general, to atherosclerosis and, in particular, to a method of treating atherosclerosis and to cells suitable for use in such a method.

This application claims priority from U.S. Provisional Application No. 60/795,203, filed Apr. 27, 2006, and U.S. Provisional Application No. 60/854,695, filed Oct. 27, 2006, the entire contents of these applications are incorporated herein by reference.

This invention was made with government support under 1RO1 AG 023073-01 awarded by the National Institutes of Health. The government has certain rights in the invention.

### TECHNICAL FIELD

The present invention relates, in general, to atherosclerosis and, in particular, to a method of treating atherosclerosis and to cells suitable for use in such a method.

### BACKGROUND

The response-to-injury hypothesis presupposed that atherosclerosis was a chronic inflammatory process following localized injury to the vessel wall, in particular to the endothelial layer lining the lumen of the vessel. Recent evidence, however, indicates that the homeostasis of the arterial wall depends on the balance between vascular injury and repair and that endothelial progenitor cells (EPCs) originating from the bone marrow contribute to the vascular repair process, accelerating reendothelialization and limiting atherosclerotic lesion formation (Asahara et al, Science 275:964-967 (1997), Goldschmidt-Clermont and Peterson, Sci. Aging Knowledge Environ. 2003 Nov. 12; 2003(45):re8). Indeed, EPC-mediated vascular repair has been demonstrated in acute vascular injury and atherosclerosis (Assmus et al, Circulation 106:3009-3017 (2002), Kawamoto et al, Circulation 103:634-637 (2001), Rauscher et al, Circulation 108:457-463 (2003)). Using genetically marked mice as donors, EPCs have been shown to engraft the injured vascular sites and differentiate into endothelial cells (Rauscher et al, Circulation 108:457-463 (2003), Sata et al, Nat. Med. 8:403-409 (2002), Luttun et al, Nat. Med. 8:831-840 (2002)). These observations have generated excitement about the possible use of bone marrow cells as a novel preventative and/or treatment strategy for atherosclerosis.

On the other hand, atherosclerosis risk factors, such as aging and diabetes, reduce the number and functional activity of EPCs. A strong inverse correlation between the number of circulating EPCs and the combined Framingham risk factor score for atherosclerosis was demonstrated (Hill et al, N. Engl. J. Med. 348:593-600 (2003)). Measurement of flow-mediated brachial-artery reactivity revealed a significant relation between endothelial function and the number of progenitor cells. The levels of circulating EPCs were a better predictor of vascular reactivity than was the presence or absence of conventional risk factors (Hill et al, N. Engl. J. Med. 348:593-600 (2003)). Furthermore, factors that reduce cardiovascular risk, such as statins or exercise, elevated EPC levels, contributing to enhanced endothelial repair (Llevadot et al, J. Clin. Invest. 108:399-405 (2001), Walter et al, Circulation 105 :3017-3024 (2002), Laufs et al, Circulation 109 :220-226 (2004)). Remarkably, it was demonstrated that injection of unfractionated bone marrow cells isolated from age-matched wild type (WT) or young, but not old, apoE^{-/-} donor mice substantially retarded the formation of atherosclerotic lesions (Rauscher et al, Circulation 108:457-463 (2003)). These data suggest that aging, particularly in the presence of atherosclerosis risk factors, may result in the exhaustion of supply of competent EPCs in the bone marrow, which may undermine the efficacy of certain cell-based therapeutic approaches, especially when autologous bone marrow cells are isolated and simply injected back into the patients. Importantly, the findings underscore the essentiality of understanding the bone marrow biology to vascular biologists and clinicians.

Indeed, the lack of comprehensive insights in the bone marrow biology represents a major challenge to clinicians and vascular biologists before performing clinical trials to evaluate cell therapy. Identification of effector EPCs and necessary supporting cells will help improve the efficacy of cell-based treatments and reduce the recruitment of mature inflammatory cells or precursors destined for hematopoietic lineages.

The present invention results from studies designed to elucidate the numerical and functional changes underlying the efficacy difference between young and old bone marrow. The invention provides, at least in part, a population of cells enriched for EPCs and therapeutic strategies based on the use of same.

### SUMMARY OF THE INVENTION

The present invention relates generally to atherosclerosis. More specifically, the invention relates to a method of treating atherosclerosis and to cells suitable for use in such a method.

Objects and advantages of the present invention will be clear from the description that follows.

### BRIEF DESCRIPTION OF THE DRAWINGS

FIGS. 1A-1L. Endothelial differentiation of lin−/cKit+/Sca-1+ HSCs at a single-cell level. A single lin−/cKit+/Sca-1+ HSC was placed in 96-well plate containing serum free Xvivo15 medium supplemented with 5×10⁻⁵ M β-mercaptoethanol, KitL (30 ng/ml), bFGF (10 ng/ml), IL-6 (10 ng/ml), Tpo (100 ng/ml) (FIG. 1A, day 0). Cell division (FIG. 1B, day 1) and colony formation (FIG. 1C, day 7) are observed. When the cells were switched to differentiation EGM medium supplemented with KitL (30 ng/ml), Flt3 ligand (Flt3-L, 30 ng/ml), IL-6 (10 ng/ml), angiopoietin 1 (100 ng/ml) and VEGF₁₆₄ (10 ng/ml), 2.2±1.7% HSCs became endothelial-like spindle cells (FIG. 1D). A similar phenomenon was demonstrated with CD31+/VEGFR2+ cEPCs. Progenies of the single lin−/cKit+/Sca-1+ HSC were stained with mouse EC surface markers VEGFR2 and Ac-LDL uptake (FIG. 1E and FIG. 1I, red color) and VWF and lectin (FIG. 1F and FIG. 1J, green color). FIG. 1G and FIG. 1K represent Hochest 33342 staining (blue color) for nuclei. FIG. 1H and FIG. 1L are the overlay of the three colors.

FIGS. 2A-2D. FACS analysis of simple little cells. Simple little cells (SLCs)—ells located in the left lower quadrant of FSC/SSC flow cytometry plot—are abundant in young (3-week old) apoE^{-/-} bone marrow (FIG. 2A). A marked reduction of SLCs is detected in the marrow of old (6-month old, fed high fat, high cholesterol) apoE^{-/-} mice (FIG. 2B). Similarly, there is a large number of SLCs in young (3-week old) WT bone marrow (FIG. 2C), compared to its old counterpart (2-year-old, FIG. 2D).

FIGS. 3A-3F. Age-dependent selective depletion of precursor cells from the SLC population. FACS analysis of young SLCs reveals that the population (FIG. 3A) is mainly composed of lin⁻ precursors (FIG. 3B), and most of these progenitors cells are cKit and Sca-1 negative (FIG. 3C). In contrast, the old SLC population (FIG. 3D) contains markedly decreased number of lin⁻/cKit⁻/Sca-1⁻ cells (FIG. 3E), whereas the relative abundance of CD31+ and lin−/cKit+/Sca-1+ cells remains unaffected by aging (FIG. 3F)

FIGS. 4A-4D. Endothelial differentiation of lin⁻/cKit⁻/Sca-1⁻ SLCs at a single-cell level. A single GFP⁺/lin−/cKit⁻/Sca-1⁻ SLC was placed in 96-well plate coated with OP9 stromal cells in MEM medium supplemented with 20% FBS, 1% PSA and 2-mercaptoethanol (10⁴ M), IL-3 (20 ng/ml), SCF (20 ng/ml), IL6 (20 ng/ml) and VEGF (10 ng/ml) (FIG. 4A, day 0). Cell division (FIG. 4B, day 1) and colony formation (FIG. 4C, day 14) are observed. GFP cells sorted by FACS from the coculture system form vascular tubes when replated and further cultured on matrigel for 8 hours (FIG. 4D).

FIGS. 5A-5L. Phenotypic characterization of lin⁻/cKit⁻/Sca-1⁻ SLC progeny. The endothelial identity of cells derived from a single GFP⁺/lin⁻/cKit⁻/Sca-1⁻ SLC (FIG. 5A and FIG. 5E, green color) is determined by the expression of endothelial surface markers CD31 and VEGFR2 (FIG. 5B and FIG. 5F, red color); FIG. 5C and FIG. 5G represent Hochest 33342 staining (blue color) for nuclei. FIG. 5D and FIG. 5H are the overlay of the three colors, revealing the expression of CD31 and VEGFR2 in virtually all GFP⁺ progeny. The excess nuclei are due to the presence of GFP negative OP-9 stromal cells. Cultured GFP+ SLCs incubated with rat anti-mouse IgG instead of rat anti-mouse VEGFR2 primary antibody followed by Alexa Fluor 594 donkey anti-rat IgG (red) staining serves as negative control for VEGFR2 (FIG. 5I to FIG. 5L) and CD31.

FIG. 6. Decrease in the number of SLCs in relation to aging, apoE deficiency and atherogenic diet. The number of SLCs is determined in the bone marrow of 3-wk-old WT and apoE^{-/-} mice, WT mice fed regular chow (age: 6 months, 1 year and 2 years), apoE^{-/-} mice fed regular chow (age: 6 months and 1 year), and apoE^{-/-} mice fed high-fat, high cholesterol diet (age: 6 months). A graded decrease in the number of SLCs is observed. *: p<0.05 compared with 3-wk-old mice of the same genotype; **: p<0.01 compared with 3-wk-old mice of the same genotype.

FIGS. 7A and 7B. Enhanced CD31 and VEGFR2 expression in GFP⁺/lin⁻/cKit⁻/Sca-1⁻ SLC progeny. RNA was prepared from single GFP⁺/lin⁻/cKit⁻/Sca-1⁻ SLC derived CD31+ and VEGFR2+ progenies (n=5) and freshly isolated whole bone marrow cells (n=4) and analyzed for CD31 and VEGFR2 expression by Taqman real-time RT-PCR. The results represent fold changes obtained from duplicate experiments and normalized by 18s rRNA expression. A substantial increase in mouse CD31 (FIG. 7A) and VEGFR2 (FIG. 7B) mRNA expression in GFP⁺/lin⁻/cKit⁻/Sca-1⁻ SLC progenies is noted as compared with uncultured whole bone marrow cells.

### DETAILED DESCRIPTION OF THE INVENTION

The present invention results, at least in part, from the identification of a group of cells characteristically located in the lower left quadrant of a forward scatter (FSC)/side scatter (SSC) flow cytometric plot that is markedly decreased in marrows from old WT and apoE^{-/-} mice but that is abundantly present in young WT and apoE^{-/-} bone marrow. These cells are termed “simple little cells” or “SLCs” because of their modest sideward and forward light scattering properties in flow cytometry, indicative of limited granular content, and small size, respectively. Analysis of SLCs reveals that the majority of these cells fall into the lineage negative, cKit negative and Sca-1 negative (lin⁻/cKit⁻/Sca-1⁻) population. The lin⁻/cKit⁻/Sca-1⁻ SLCs differentiate into mature endothelial cells (ECs) more efficiently than other bone marrow fractions, including lin⁻/cKit⁺/Sca-1⁺ hematopoietic stem cells (HSCs) and CD34⁺/VEGFR2⁺ conventional EPCs (cEPCs). These data indicate that after a lifetime of repairing atherosclerotic arteries, the supply of the specific type(s) of EPC(s), needed to maintain the homeostasis of the cardiovascular system, becomes exhausted.

Thus, the present invention is based, at least in part, on the identification of cells that account for the anti-atherosclerotic effects exerted by young bone marrow cells. The present invention provides a method of attenuating atherosclerosis progression, even in the continued presence of vascular injury. In accordance with this method, vascular repair/rejuvenation is effected using endothelial/vascular progenitor cell engraftment.

Cells suitable for use in the present invention include endothelial progenitor cells, that is, stem cells capable of maturing at least into mature endothelial cells (e.g., vascular endothelial cells). Suitable progenitor cells can be isolated from embryos and from hematopoietic and stromal fractions of bone marrow (Reyes et al, Blood 98:2615-2625 (2001), Sata et al, Nat. Med. 8:403-409 (2002)). Suitable progenitor cells can also be isolated from peripheral blood (or umbilical cord blood); advantageously, the cells are derived from young, non-atherosclerotic mammals (e.g., humans).

Populations of cells significantly enriched in specific cell lineages having a propensity for vascular repair/rejuvenation are described in the Example that follows and are preferred for use in the present method. These cells are, advantageously, lin-SLCs, preferably, lin⁻/cKit⁻/Sca-1⁻ SLCs.

Methods of isolating progenitor cells are well known in the art (see, for example, Reyes et al, J. Clin. Invest. 109:337 (2002), Reyes et al, Blood 96:2615-2625 (2001), Sata et al, Nat. Med. 8:403-409 (2002), U.S. Pat. No. 5,980,887 and US Patent Appln. 20020051762). The Example that follows describes a method of isolating SLCs, including lin⁻/cKit⁻/Sca-1⁻ SLCs.

Autologous or heterologous cells can be used in accordance with the invention and can be expanded in vivo or ex vivo prior to administration. Expansion can be effected using standard techniques (see, for example, U.S. Pat. No. 5,541,103).

Suitable cells, e.g., lin⁻/cKit⁻/Sca-1⁻ SLCs, can be administered using any of a variety of means that result in vascular distribution (e.g., via catheter or via injection), injection of the cells intravenously being preferred. The optimum number of cells to be administered and dosing regimen can be readily determined by one skilled in the art and can vary, for example, with the patient status and the effect sought.

Engraftment of the cells described herein can be used prophylactically or therapeutically, alone or in combination with other approaches designed to prevent atherosclerosis or to attenuate atherosclerotic progression. In this regard, the progenitor cells of the invention can be manipulated (e.g., prior to administration) to serve as carrier or delivery vehicles of agents that have a therapeutic (e.g., anti-atherosclerotic) effect. Such agents can be proteinaceous or non proteinaceous.

For example, the progenitor cells can be used as vehicles for gene delivery. In accordance with this aspect of the invention, a recombinant molecule comprising a nucleic acid sequence encoding a desired protein, operably linked to a promoter, can be delivered to a vascular site (e.g., an atherosclerotic site). The recombinant molecule can be introduced into the progenitor cells using any of a variety of methods known in the art. An effective amount of the transformed progenitor cells can then be administered under conditions such that vascular distribution is effected, expression of the nucleic acid sequence occurs and production of the protein product results. The recombinant molecule used will depend on the nature of the gene therapy to be effected. Vectors suitable for use in endothelial progenitor cells are well known in the art, as are methods of introducing same into progenitor cells (see, for example, U.S. Pat. No. 6,878,371). Promoters can be selected so as to allow expression of the coding sequence to be controlled endogenously (e.g., by using promoters that are responsive to physiological signals) or exogenously (e.g., by using promoters that are responsive to the presence of one or more pharmaceutical).

Any of a variety of encoding sequences can be used in accordance with this aspect of the invention. The nucleic acid can encode, for example, a product having an anti-atherosclerotic effect. For example, nucleic acids encoding proteins that afford protection from oxidative damage (such as superoxide dismutase (see, for example, U.S. Pat. No. 6,190,658 or glutathione peroxidase (see, for example, US Patent Appln. 20010029249)) can be used, as can nucleic acids encoding components in the synthetic pathway to nitric oxide (see, for example U.S. Pat. No. 5,428,070) or nucleic acids encoding agents that modulate Toll-like receptor activity (see, for example, US Patent Appln. 20030022302). Nucleic acids encoding proteins that lower total serum cholesterol, such as an apoE polypeptide (see, for example, US Patent Appln. 20020123093) can be used, as well as nucleic acids that encode agents that modulate expression of or activity of the products of the fchd531, fchd540, fchd545, fchd602 or fchd605 genes (see US Patent Appln. 20020102603). Nucleic acids encoding proteins suitable for use in treating inflammatory diseases can also be used, such as the glycogen synthase kinase 3β protein (see US Patent Appln. 20020077293). (See also, for example, US Patent Appln. 20010029027, 20010053769, and 20020051762 and U.S. Pat. No. 5,980,887).

The progenitor cells can also be used to administer non proteinaceous drugs to vascular sites. Such drugs can be incorporated into the cells in a vehicle such as a liposome or time released capsule.

In addition to the use of endothelial progenitor cells as of delivery vehicles for proteinaceous and non-proteinaceous therapeutics, the progenitor cells can also be used to deliver non-therapeutic agents to the vessel wall. Such agents include imaging agents (e.g., MRI imaging agents), such as nano- and micro-particles of iron (e.g., Feridex) and other superparamagnetic contrast agents. The use of such labeled progenitor cells permits monitoring of cellular biodistribution over time. Methods of introducing such agents are known in the art (see, for example, Bulte et al, Nat. Biotechnol. 19:1141-1147 (2001), Lewin et al, Nat. Biotechnol. 18:410-414 (2000), Schoepf et al, BioTechniques 24:642-651 (1998), Yeh et al, Magn. Reson. Imaging 30:617-625 (1997), Lewin et al, Nat. Biotechnol. 18:410-414 (2000), Schoepf et al, BioTechniques 24:642-651 (1998), Yeh et al, Magn. Reson. Imaging 30:617-625 (1997), Frank et al, Acad. Radiol. 9:5484-5487 (2002)). Cell administration methods such as those described above can be used.

The results provided in the Example that follows indicate that injecting (or otherwise administering) SLCs, advantageously, lin⁻/cKit⁻/Sca-1⁻ SLCs, can result in vascular repair without the introduction of inflammatory cells within the vessel wall, which can further aggravate the inflammatory process associated with atherosclerosis.

It will be appreciated from a reading of this disclosure that the SLCs described herein may encompass several cell types. For example, certain cell types may serve as true EPCs or smooth muscle progenitors, whereas other cells, acting as supporting cells, may influence the differentiation of EPCs or smooth muscle progenitors, for example, by secreting paracrine factor. Hence, SLCs may offer better efficacy, as compared with purified progenitor subtypes. The SLCs described herein can also be advantageous over other candidate cells (purified or non-purified), identified by the expression of specific cell surface markers in that SLCs can be easily isolated without the use of biochemical reagents to tag these cells—which reagents can adversely affect progenitor cell survival, proliferation and differentiation.

In view of the fact that decreased circulating EPC levels can be predictive of atherosclerosis disease outcome, the present invention includes diagnostic methods designed to identify high risk patients based on low levels of EPCs.

Furthermore, since patients most in need of EPCs may not possess sufficient numbers for autologous transplantation, the invention also includes methods of enhancing EPC numbers or function.

As will also be appreciated from a reading of this disclosure, the present approaches have applicability in human and non-human animals.

Certain aspects of the invention are be described in greater detail in the non-limiting Example that follows (see also Rauscher et al, Circulation 108:457-463 (2003) and Goldschmidt-Clermont et al, SAGE KE Nov. 12, 2003, pp. 1-5 (http://sageke.sciencemag.org/cgi/content/full/sageke;2003/45/re8)). This application is related to U.S. Provisional Application No. 60/795,203, filed Apr. 27, 2006, and to U.S. application Ser. No. 10/788,423, filed Mar. 1, 2004, the entire contents of which are incorporated herein by reference.

### EXAMPLE

### Experimental Details

Animals

ApoE^{-/-} C57BL/6J mice and Wild-type (WT) C57BL/6 mice (6-8 weeks of age) were purchased from The Jackson Laboratory (Bar Harbor, Me.). EGFP-expressing C57BL/6 mice were provided by Dr. Nelson J Cao, Division of Cellular Therapy, Duke University Medical Center. Pups were weaned at 3 weeks (wks) of age and fed either regular chow or a Western diet #88137 (Harlan-Teklad; 42% fat, 1.25% cholesterol, Teklad, Madison, Wis.). Animals were housed in sterile microisolator cages in which they received autoclaved food and autoclaved acidified drinking water in a specific pathogen-free facility throughout the study. Mice were euthanized at the age of 3 wks, 6 months, 1 year or 2 years. At the time of euthanization, bone marrow cells were collected. Studies were performed in accordance with Duke University Institutional Animal Care and Use Committee-approved procedures.

Cell Characterization and Fluorescence Activated Cell Sorting (FACS) Analysis

Bone marrow was isolated from wild type, apoE^{-/-}, and EGFP positive mouse tibiae and femora. Single-cell suspensions were obtained in staining medium (HBSS+3% fetal calf serum, 10 mM Hepes, PH 7.0, and 2 mM EDTA) after ammonium chloride lysis of the red blood cells and straining through a nylon mesh. Bone marrow cells were stained with biotinylated monoclonal antibodies to the following mouse cell surface antigens: CD3 (KT31.1), CD4 (GK1.5), CD5 (53-7.8), CD8 (53-6.7), CD45R/B220 (6B2), erythroid cells (Ter119), Mac-1 (M1/70), and 8C5 (Gr-1) followed by incubation with antibiotin Streptavidin PE-Texas Red. The gating of the lineage negative (lin−) versus lineage positive (lin+) cells was determined using parameter histograms that display the relative fluorescence plotted against the number of events. The line separating lin− from lin+ cells was placed in between the peaks representing the two cell populations. The gating was further aided with the use of negative control (biotinylated IgG isotype control) and positive control (peripheral blood mononuclear cells stained with lineage cocktail). Hematopoietic stem cells (HSCs) were identified using PE-E13-161-7 (anti-Sca-1), allophycocyanin (APC)-2B8 (anti-c-Kit) antibodies in the lin− population. Conventional endothelial progenitor cells (cEPCs) were detected with a PE-conjugated rat anti-mouse VEGFR2 antibody and FITC-conjugated rat anti-mouse CD34 antibody. All rat antibodies were obtained from BD-Pharmingen. Labeled cells were detected with a dual laser FACS Vantage™ (Becton Dickinson) flow cytometry. Dead cells were excluded from analysis using propidium iodide staining. For the identification of lin−/cKit−/Sca-1− SLCs, unfractionated bone marrow mononuclear cells were utilized. SLCs were selected first on the FSC/SSC flow cytometric plot, followed by lineage negative and CD31 negative cells, which were further gated for Sca-1 and c-Kit expression. The gating of lin− versus lin+ SLCs was performed in the same fashion as described above for the whole bone marrow cells. Flow cytometry data were analyzed using FlowJo software (Treestar Inc.), and two parameter data are presented as 5% probability plots with outliers.

En Masse Cell Culture

Lin⁻/cKit⁻/Sca-1⁻ SLCs, lin⁻ whole bone marrow fraction minus SLCs (WB-SLCs), CD34⁺/VEGFR2⁺ cEPCs and lin−/cKit⁺/Sca-1⁺ HSCs were plated on 24-well plate coated with fibronectin at the density of 10⁴/well in 500 μl expansion medium composed of endothelial basal medium-2 (EBM, Clonetics) supplemented with 20% fetal bovine serum, 1% penicillin/streptomycin and mouse cytokines: 50 ng/mL SCF, 50 ng/mL class III receptor tyrosine kinase ligand (Flt3-L), 10 ng/mL IL-3, 100 ng/ml angiopoietin I, 10 ng/ml VEGF and 10 ng/mL IL-6, 10 ng/ml EGF, 10 ng/ml IGF-1 and 10 ng/ml bFGF (Biosource International, Rockville, Md.). After two wks, EGF, IL-3 and Flt3-L were withdrawn from the expansion medium to induce EC differentiation. The plating efficiency/colony forming units (CFUs) were counted under a phase-contrast microscope after 14 days of incubation at 37° C. in a humidified atmosphere of 5% CO2. The endothelial identity of the cultured cells was determined by fluorescence staining as described below for the expression of EC specific markers.

Single-Cell Culture

HSCs and cEPCs: Single Lin^{neg}Sca-1^{pos}-Kit^{high} HSC or CD34+/VEGFR2+ cEPC derived by two successive sorts was deposited per well in 96-well plates (Corn) using the single cell deposition unit and FACS Diva software on a FACS Vantage SE multi-color high-speed sorter. The wells contained 100 μl of serum-free medium (Xvivo15; BioWhittaker) supplemented with 10% fetal bovine serum, penicillin (100 U/mL), streptomycin (100 μg/mL), 5×10⁻⁵ M β-mercaptoethanol, IL-3 (20 ng/ml), IL-6 (10 ng/ml), SCF (30 ng/ml), Flt3 (100 ng/ml), Flt3 ligand (Flt3-L, 30 ng/ml), thrombopoietin (Tpo, 100 ng/ml), Angiopoietin I (100 ng/ml), basic fibroblast growth factor (bFGF, 100 ng/ml), epidermal growth factor (EGF, 100 ng/ml), and vascular endothelial growth factor (VEGF10 ng/ml). All factors were purchased from R&D Systems. After two weeks, a colony was formed from a single HSC in the center of well. Cells were then passaged into 8-well chamber slides containing EGM plus medium to induce EC differentiation. After 4 or 6 weeks, cells were identified with VEGF, CD31 staining and Ac-LDL uptake.

SLCs: An OP9 mouse stromal cell (American Type Culture Collection, Manassas, Va.) layer was prepared in 96-well microtiter plate in OP9 growth medium consisting of a-modified minimum essential media (α-MEM) supplemented with 20% FBS, 1% PSA and 2-mercaptoethanol (10⁻⁴ M). A single GFP positive Lin⁻/cKit⁻/Sca-1⁻ SLC, or WB-SLC cell, was sorted out and deposed onto each well with pre-cultured OP9 stromal cell layer using Clone Cyt (Becton Dickinson) apparatus of FACS Vantage and subjected to in vitro differentiation assay. Recombinant mouse IL-3 (20 ng/ml), SCF (20 ng/ml), IL6 (20 ng/ml) and VEGF (10 ng/ml) were added in the culture medium. For each cell type, at least 192 wells (2×96-well plates) per mouse and from five mice per group were used. The progeny were either stained directly with EC-specific markers as described below to establish their endothelial identity or harvested and stained with APC-conjugated rat anti-mouse CD31 and PE-conjugated rat anti-mouse VEGFR2 antibody. CD31 or VEGFR2 positive cells were sorted out by flow cytometry and analyzed for expression of GFP. GFP⁺/CD31⁺/VEGFR2⁺ cells within the lin⁻/cKit⁻/Sca-1⁻ SLC fraction were subjected to further culture for induction of capillary-like tube formation and for gene expression study at the RNA level using Real time PCR.

Capillary-Like Tube Formation on Matrigel

Matrigel (BD Bioscience, Bedford, Mass.) basement membrane matrix was added to 96-well culture plates and incubated at 37° C. until gelation occurred. Sorted GFP⁺/CD31⁺/VEGFR2⁺ cells (7,000) from cultured lin⁻/cKit⁻/Sca-1⁻ SLCs were added to each well in 100 μL EGM-2 media containing 15% FBS, 1% PSA, 10 ng/mL recombinant mouse VEGF and 10 ng/ml mouse IL-6. Twelve hours later, capillary tube formation was assessed via phase contrast microscopy.

Immunofluorescence Staining

For the identification of endothelial cells derived from lin⁻/cKit⁻/Sca-1⁻ SLCs, WB-SLCs, CD34⁺/VEGFR2⁺ cEPCs, or lin⁻/cKit⁺/Sca-1⁺ HSCs, cells cultured either en masse or at a single cell level were fixed for 10 minutes in 4% paraformaldehyde in PBS and subjected to double or triple immunostaining for EC-specific markers or Dil-acetylated low-density lipoprotein (Dil-Ac-LDL) uptake. Briefly, after washing with PBS, slides were blocked for 30 minutes with 2% BSA in PBS, incubated for 1 hour with primary antibodies against CD31 (1:50), VEGFR2 (1:50) or VE-Cadherin (1:50) (R&D Systems). Secondary antibodies and Hoechst 3342 for DNA staining (Molecular Probes) were used at concentrations of 1:250 to 1:500 and incubated for 30 minutes. Between each step, slides were washed with PBS in 0.3% BSA. All procedures were performed at room temperature in dark. For Dil-Ac-LDL uptake, cells were incubated in EGM2 serum-free medium containing 10 μg/ml Dil-Ac-LDL (Molecular Probes) for 4 hours. Cells were then washed and observed by fluorescence microscopy.

TaqMan Real-Time RT-PCR (TRT-PCR)

Sorted GFP⁺/CD31⁺/VEGFR2⁺ cells isolated from lin⁻/cKit⁻/Sca-1⁻ SLC/OP9 stromal cell co-culture were further cultured for 5 days. RNA was isolated from GFP⁺/CD31⁺/VEGFR2⁺ Cells using RNeasy Mini kit (Qiagen). Freshly isolated bone marrow mononuclear cells were used as a baseline control. One μg total RNA was used for the synthesis of first strand cDNA using the SUPERSCRIPT Preamplification System (Life Technologies). PCR was optimized for the quantitation of CD31 and VEGFR2 with specific primers and probes (mCD31-forward promer, 5′-gcccaatcacgtttcagttt-3′, mCD31-reverse primer, 5′-tgtccttcctgcttcttgct-3′, mCD31-probe, 5′-6-FAM-ccttccaccaagcggtcgtg-TAMRA-3′; mVEGFR2-forward primer, 5′-atgaattgcccttggatgag-3′, mVEGFR2-reverse primer, 5′-agcgtctgcctcaatcactt-3′, mVEGFR2-probe, 5′-6-FAM-cgctgtgaacgcttgcctta-TAMRA-3′). House-keeping gene 18S ribosomal RNA was used as an internal control. A sequence detector (ABI Prism 7700, PE Applied Biosystems) was used to measure the amplified product in direct proportion to the increase in fluorescence emission continuously during the PCR amplification. For each sample, a threshold cycle (Ct) value was calculated from each amplification plot, representing the PCR cycle number at which the fluorescence was detectable above an arbitrary threshold. To normalize Ct of the target gene copies to 18S rRNA, ΔCt was calculated as Ct (target)−Ct (18S rRNA). Data presented were derived from ΔCt and expressed as the mean of 5 independent experiments, with each assayed in duplicate.

Statistical Methods

All flow cytometry and en masse culture data were derived from five mice per group. Single cell culture data were derived from at least 192 cells per mouse and 5 mice per group. TRT-PCR data were derived from GFP⁺/CD31⁺/VEGFR2⁺ cell population generated from 5 single Lin⁻/cKit⁻/Sca-1⁻ SLC cells in duplicate and freshly isolated bone marrow mononuclear cells from four mice. Group means were compared using either Students t test or ANOVA with Fisher post hoc test as appropriate. Data are presented as mean +SD with p<0.05 accepted as significant.

Results

Aging Results in Exhaustion of Simple Little Cells

It has been demonstrated previously that administration of unfractionated bone marrow from age-matched WT or young, but not old, apoE^{-/-} mice significantly reduced atherosclerotic lesion formation in apoE^{-/-} recipient mice (Rauscher et al, Circulation 108:457-463 (2003)). Thus, aging in the pro-atherogenic milieu of apoE deficiency eliminated bone marrow cell efficacy in reducing atherosclerosis. Consequently, it was reasoned that bone marrow from old apoE^{-/-} mice would be deficient, either functionally or numerically, in vascular repair-competent EPCs and perhaps the needed supporting cells. Identification of these cells, therefore, should be achievable by comparing the bone marrow from old versus young mice. FACS analysis of the young (3-wk-old, weaning) versus old (6-month-old, fed high-fat, high-cholesterol diet) apoE^{-/-} bone marrow was first focused on two types of progenitor/stem cells—EPCs and HSCs—both of which have been shown to differentiate into mature ECs, although the evidence has been conflicting for HSCs (Takakura et al, Cell 102:199-209 (2000), O'Neill et al, Circ. Res. 97:1027-1035 (2005)). cEPCs, as identified by the expression of CD34 and flk-1/KDR/VEGFR2, were present in the bone marrow of both young and old mice (accounting for 0.034±0.021% and 0.029±0.017% of the total mononuclear cells in the young and old apoE^{-/-} marrow, respectively), and there was no significant difference between these two groups (p>0.05). Similarly, lin⁻/cKit⁺/Sca-1⁺ HSCs constituted 0.067±0.035% and 0.08±0.027% of the mononuclear cells in the young and old apoE^{-/-} marrow, respectively, (p>0.05). These data indicate that atherosclerosis risk factors do not affect the numerical composition of cEPCs and HSCs in the bone marrow.

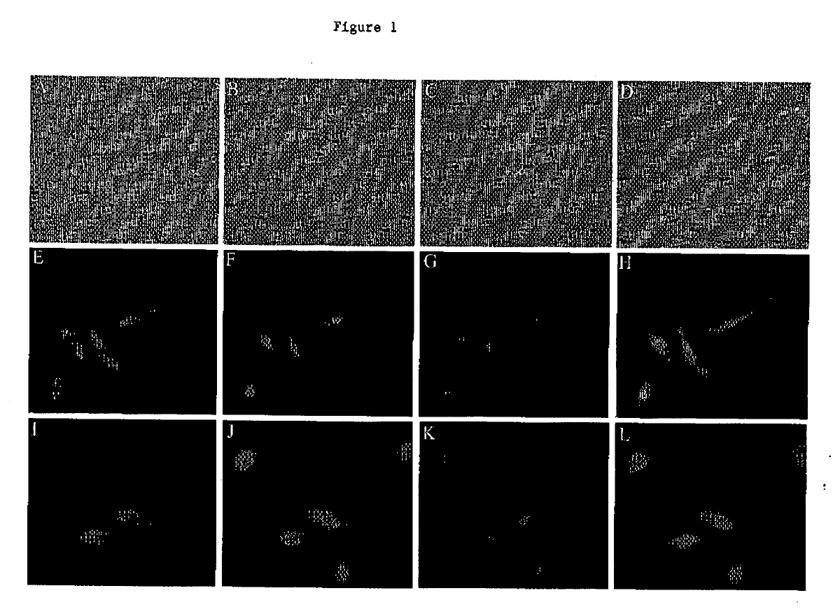

To determine if aging and atherosclerosis adversely impacted the function of cEPCs and HSCs, an examination was made of the differentiation capability of these cells isolated from young (3-wk-old, weaning) and old (6-month-old, fed high-fat, high-cholesterol diet) apoE^{-/-} bone marrow, both en masse and at the single cell level. Consistent with previous reports (Mohle et al, Blood 89:72-80 (1997)), both cEPCs and HSCs possessed the ability to convert into a mature EC phenotype, as confirmed by the positive staining for EC surface markers including VEGFR2, vWF, ulex-lectin and Dil-Ac-LDL uptake, with the plating efficiency of 2.9±1.3% for cEPCs and 2.2±1.7% for HSCs, when old and young bone marrow cells were analyzed together (FIG. 1). In addition, comparison of young versus old cEPCs and HSCs revealed that young cells were not superior to their old counterparts in acquiring an EC phenotype (3.3±1.5% versus 2.5±1.3%, p>0.05 for cEPCs, and 1.9±0.8% versus 2.3±2.1%, p>0.05 for HSCs). These findings indicate that aging and hyperlipidemia do not weaken the EC differentiation potential of cEPCs and HSCs.

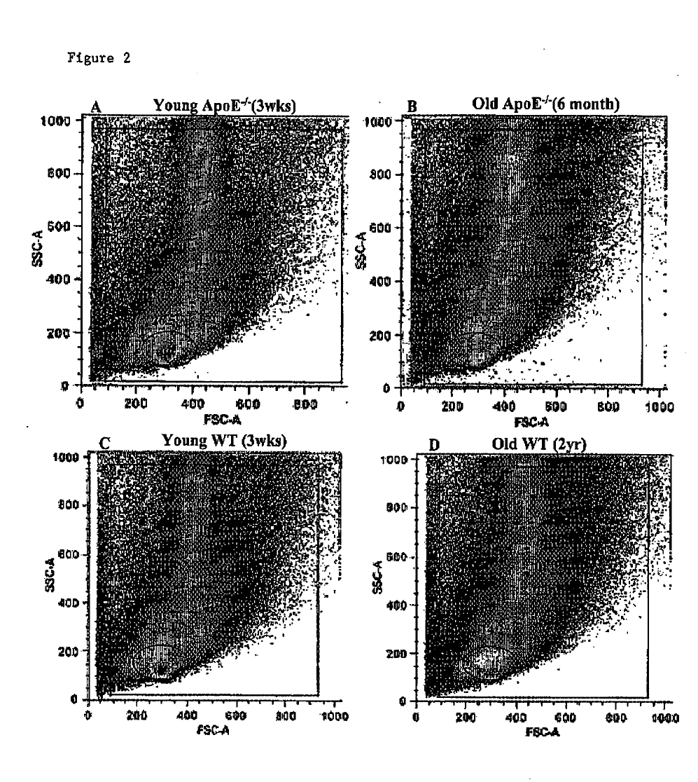

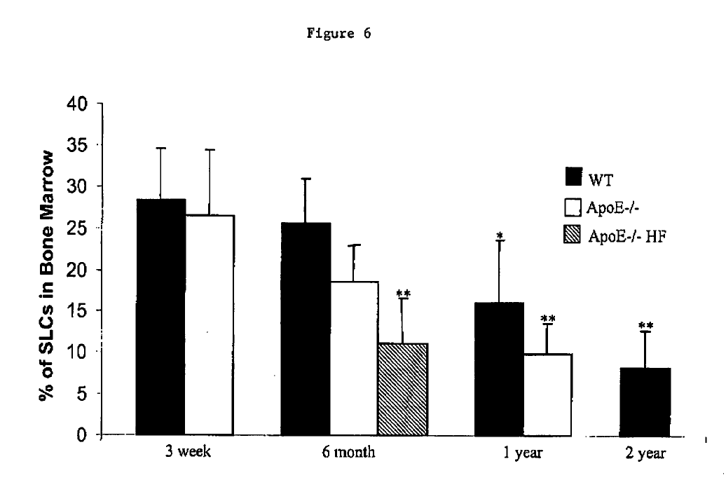

Since it is controversial regarding whether the CD34+/VEGFR2+ cEPCs represent the authentic EPCs, and there is conflicting evidence for the plasticity of HSCs, in particular in terms of their ability to differentiate into ECs in vivo (O'Neill et al, Circ. Res. 97:1027-1035 (2005)), a more inclusive approach was chosen to analyze the bone marrow—comparing forward scatter (FSC)/side scatter (SSC) flow cytometric plot of unfractionated bone marrow mononuclear cells isolated from weaning, 3-wk-old WT and apoE^{-/-} mice, WT mice fed regular chow (age: 6 months, 1 year and 2 years), apoE^{-/-} mice fed regular chow (age: 6 months and 1 year), and apoE^{-/-} mice fed high-fat, high cholesterol diet (age: 6 months). Considering that the lifespan is 2.5-3 years for WT mice, 12-14 months for apoE^{-/-} mice fed regular chow, and 8-12 months for apoE^{-/-} mice fed high-fat, high cholesterol diet, these age points represent the two extremes and the midpoint of the aging/accelerated aging process. As shown in FIG. 6, there was a graded decrease in a grouping of cells located in the left lower quadrant of the FSC/SSC flow cytometric plot relative to aging and atherosclerosis status. These cells are termed simple little cells, or SLCs, due to their modest granularity (SSC) and small size (FSC). Detailed analysis of the bone marrow revealed that the number of SLCs was equivalent in 3-wk-old weaning WT and apoE^{-/-} mice of the same age (28.4%±6.3% and 26.5±7.8%, respectively). The number of SLCs in 2-year-old WT mice (8.3±4.5%) was similar to that of 1-year-old apoE^{-/-} mice fed regular chow (9.8±3.9%) and 6-month-old apoE^{-/-} mice fed high-fat, high-cholesterol diet (11.2±5.5%), whereas there were twice as many SLCs in the bone marrow of 1-year-old WT mice (16.2±7.5%) and 6-month-old apoE^{-/-} mice fed regular chow (18.6±4.3%). The remarkable decrease of SLCs in young versus old WT and apoE^{-/-} mice fed high-fat, high cholesterol diet is further depicted in FIG. 2. These data indicate that the consumption of SLCs is age dependent and that there is an additive effect of apoE deficiency and high-fat, high-cholesterol diet on the rate of SLC consumption.

Aging Affects the Composition of Simple Little Cells

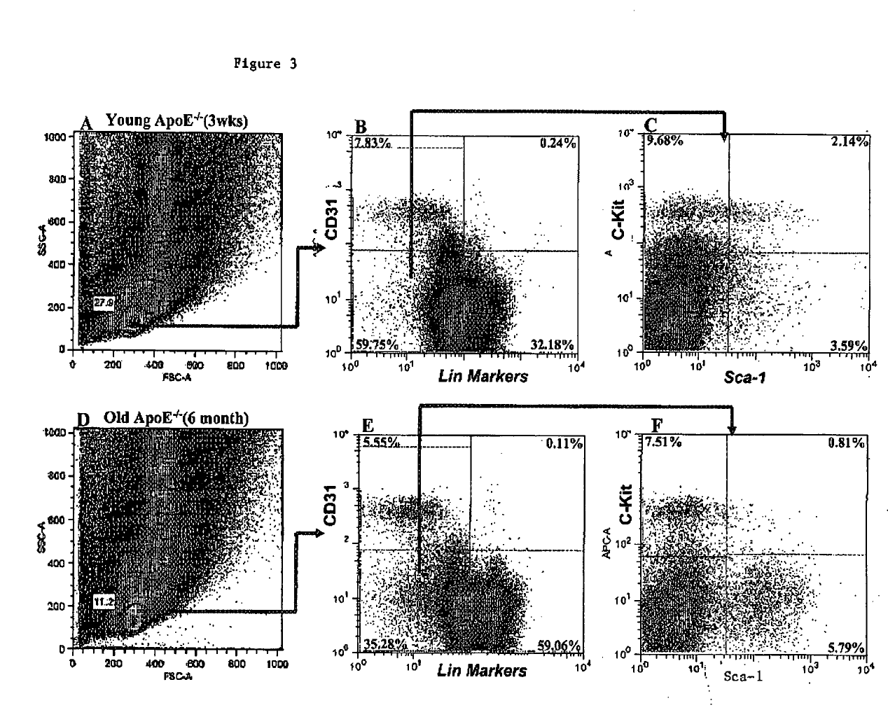

It was demonstrated that SLCs were markedly decreased with progressive aging, in particular in the presence of hyperlipidemia; the next question addressed was what cells constituted the SLC population. The focus was on the young bone marrow, where SLCs were abundant. FACS analysis of the SLC fraction in 3-wk-old apoE^{-/-} mice using lineage cocktails (markers for mature hematopoietic cells) and CD 31 (mature EC marker) showed that most of the cells were lineage low/negative (lin^{-/low}) and CD31 negative, indicating that the young SLC population is enriched for immature precursors. When the lin⁻/CD31⁻ fraction was further analyzed for the expression of cKit/Sca-1, surprisingly, most of the cells were cKit and Sca-1 negative (FIGS. 3A-3C).

A determination was then made to whether aging would result in exhaustion of selected cell types from the SLC population, in addition to decreasing the total number of SLCs. Comparison of young (3 weeks) with old (6 months, high-fat, high-cholesterol diet) apoE^{-/-} bone marrow revealed that the percentage of lin⁻ precursors dropped to from 58.3±7.4% to 39.6±10.2% (p<0.01), whereas lin⁺ mature hematopoietic cells increased from 37.5±8.5% to 66.7±3.4% (p<0.01) in the SLC fraction (FIGS. 3D-3F). The increase in lin⁺ cells in the SLC population is consistent with an increased granularity in the bone marrow as a whole (FIGS. 2B, 2D and 3D), which may represent increased proportion of neutrophils and decreased fraction of B cells with aging. Instructively, the percentages of CD31+ ECs remained unchanged. Furthermore, the relative abundance of cKit⁺/Sca-1⁺ HSCs did not differ between young and old bone marrow within the lin⁻ SLC fraction (FIGS. 3D-3F). Thus, lin⁻/cKit⁻/Sca-1⁻ cells were the subpopulation most affected by aging. These data indicate that aging not only decreases total SLCs, but also selectively depletes immature precursors, in particular the lin⁻/cKit⁻/Sca-1⁻ subpopulation, within the SLC population.

Lin−/cKit−/Sca-1− Simple Little Cells Differentiate into Mature Endothelial Cells

Since lin⁻/cKit⁻/Sca-1⁻ SLCs represent the cell type that is affected most profoundly by aging and atherosclerosis and both of which are associated with decreased bone marrow efficacy, an investigation was made to determine if these cells could adopt a mature EC phenotype under conditions in favor of EC differentiation in vitro. When 10⁴ lin⁻/cKit⁻/Sca-1⁻ SLCs were plated in 24-well plate, a fraction of the cells were capable of unlimited self-replication, which further formed colonies. When counted two weeks after seeding, the colony forming efficiency (CFE) was 9.6±3.1% for young WT cells. The lin⁻ fraction of the bone marrow mononuclear cells depleted of SLCs (WB-SLCs) was also cultured. The CFE for these cells was 1.9±2.1% (p<0.01). Immunofluorescence examination of the SLC progeny revealed that these cells expressed markers for mature ECs, including CD31, VEGFR2, VE-Cadherin, and were capable of AcLDL-DiI uptake (data not shown).

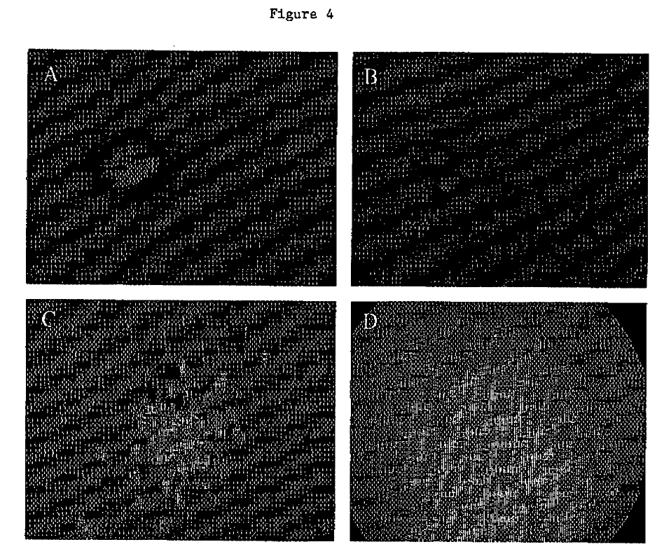

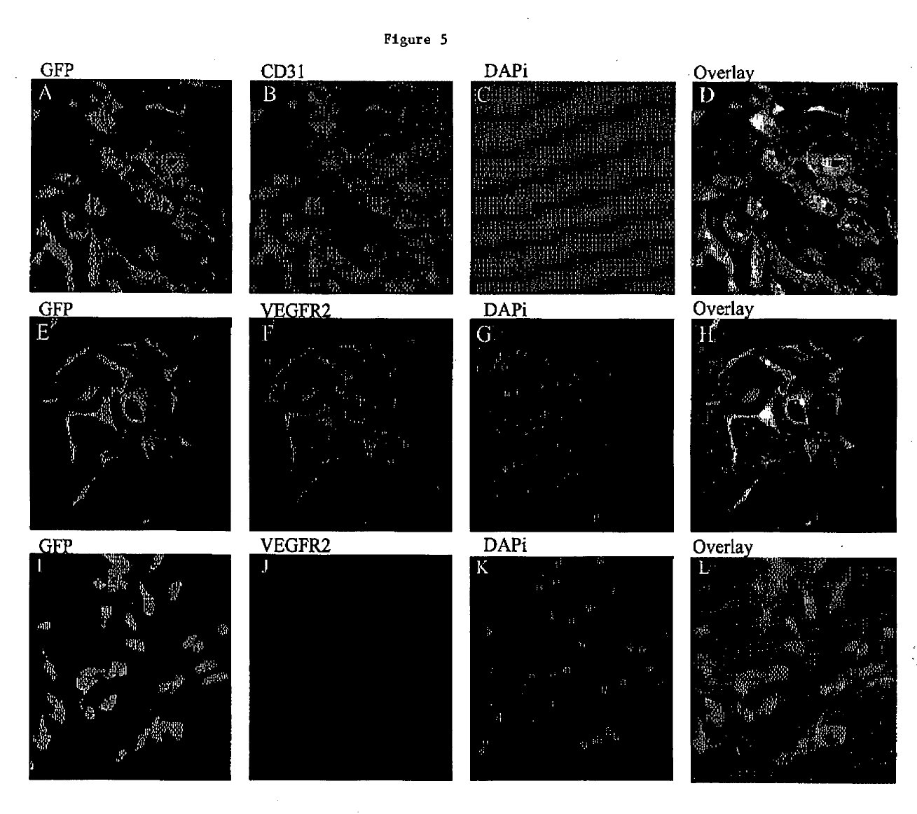

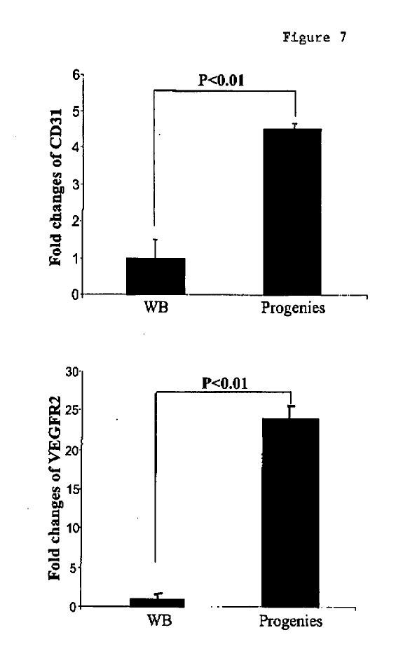

To further confirm that the lin⁻/cKit⁻/Sca-1⁻ SLC population was indeed enriched for progenitors that were capable of converting to mature ECs, single-cell culture conditions for these cells were optimized, which, in contrast to cEPC and HSC single cell culture, required the presence of OP-9 stromal cells as feeders. Thus, lin⁻/cKit⁻/Sca-1⁻ SLCs were isolated from 3-wk-old EGFP mice and these cells were cultured at a single-cell level together with WT OP-9 stromal cells. Indeed, a portion of these cells formed colonies (FIGS. 4A-4C). The SLC progeny formed vascular tubes when cultured on matrigel (FIG. 4D). Furthermore, progeny of single lin⁻/cKit⁻/Sca-1⁻ SLCs stained positive for VEGFR2 and CD31 (FIGS. 5A-5H). To confirm the immunofluorescence data, TRT-PCR analysis was performed for CD31 and VEGFR2, which revealed substantially increased CD31 and VEGFR2 mRNA expression in the progeny of single lin⁻/cKit⁻/Sca-1⁻ SLCs compared to uncultured whole bone marrow cells (FIG. 7). Consistent with the CFE data obtained in en masse culture, the plating efficiencies for these cells were 7.4±2.3% for lin⁻/cKit⁻/Sca-1⁻ SLCs, and 2.1±1.8% for WB-SLCs. Collectively, these data indicate that lin⁻/cKit⁻/Sca-1⁻ SLC fraction, the supply of which is exhausted with aging and atherosclerosis, is enriched for progenitors that are capable of adopting a mature EC phenotype in vitro. It is noteworthy that the plating efficiency for the lin⁻/cKit⁻/Sca-1⁻ SLCs is much higher than that for cEPCs (2.9±1.3%) and HSCs (2.2±1.7%).

The EC differentiation efficiency of old lin⁻/cKit⁻/Sca-1⁻ SLCs was then examined by using 2-year-old GFP bone marrow whose number of SLCs was equivalent to 6-month-old apoE^{-/-} mice fed high-fat diet, high-cholesterol diet. The plating efficiency for these cells was 6.3±2.7% for en masse culture and 4.1±2.3% for single cell culture. The difference in plating efficiency between young and old lin⁻/cKit⁻/Sca-1⁻ SLCs was borderline significant (p=0.051 for en masse and p=0.053 for single cell culture). Although it was not possible to use lin⁻-cKit⁻/Sca-1⁻ SLCs isolated from young and old apoE^{-/-} mice to study the combined effects of aging, apoE deficiency and high-fat diet, high-cholesterol diet on the differentiation capability of these cells due to the difficulty to distinguish apoE^{-/-} lin⁻/cKit⁻/Sca-1⁻ SLC progeny from OP-9 feeder cells, the findings indicate that additional work, particularly in vivo experiments, is warranted to fully characterize the impact of aging and atherosclerosis on the function of lin⁻/cKit⁻/Sca-1⁻ SLCs.

Summarizing, the identification of EPCs extracted from human peripheral blood in 1997 by Asahara et al, Science 275:964-967 (1997) inspired substantial efforts to investigate the mechanisms that maintain and restore endothelial integrity and function, the disruption of which represents a critical event in atherogenesis. Clinical studies have shown that traditional risk factors for atherosclerosis are associated with low levels of circulating EPCs (Hill et al, N. Engl. J. Med. 348:593-600 (2003), Heiss et al, J. Am. Coll. Cardiol. 45:1441-1448 (2005, Fadini et al, J. Am. Coll. Cardiol. 45:1449-1457 (2005), Dong et al, J. Am. Coll. Cardiol. 45:1458-1460 (2005)), whereas factors that reduce cardiovascular risk, such as statin therapy or exercise appear to elevate EPC levels (Lievadot et al, J. Clin. Invest. 108 :399-405 (2001), Walter et al, Circulation 105 :3017-3024 (2002), Laufs et al, Circulation 109 :220-226 (2004)). Recently, Werner and colleagues (Werner et al, N. Engl. J. Med. 353:999-1007 (2005)) found that higher levels of EPCs were associated with a reduced risk of death from cardiovascular causes and of the composite end point of major cardiovascular events, after adjustment for traditional risk factors and prognostic variables. Hill et a, N. Engl. J. Med. 348:593-600 (2003) found that even in healthy subjects, the levels of EPCs were inversely correlated with the combined Framingham risk factor score for atherosclerosis and predicted vascular function better than the Framingham risk score. Numerous animal studies have shown that EPCs participate not only in forming new blood vessels but also in maintaining the integrity and function of vascular endothelium (Asahara et al, Science 275:964-967 (1997), Rauscher et al, Circulation 108:457-463 (2003), Takahashi et al, Nat. Med. 5:434-438 (1999)). It has been demonstrated that repeated injection of bone marrow-derived cells into atherosclerosis-prone apoE^{-/-} mice reduced the rate of plaque formation without altering serum lipids levels, and that donor EPCs engrafted and differentiated into ECs in the recipient's blood vessels (Rauscher et al, Circulation 108:457-463 (2003)). These studies have provided insights into the vascular repair mechanisms and basis for the development of new therapeutic approaches involving bone marrow cells.

Due to the lack of understanding of bone marrow biology, most preclinical and clinical studies have been based on introducing either whole bone marrow cells or a crude bone-marrow cell population potentially containing EPCs, hematopoietic cells and irrelevant pluripotent cells, with some animal experiments using either purified conventional EPCs, such as CD133+/CD34+ or CD34+/VEGFR2+ cells, or CD34+ hematopoietic stem cells. The use of whole bone marrow or crude cell preparations risks the introduction of nonessential, and at time noxious, cells into the vessel wall, which may be associated with increased toxicity; for example, injection of monocytic precursors may result in accumulation of inflammatory cells within the vessel wall, further aggravating the inflammatory process associated with atherosclerosis. The isolation and characterization of EPCs have been confounded by the lack of specific endothelial markers on angioblast-like progenitors and assays to distinguish EPCs from mature ECs sloughed from the vessel wall, and from hematopoietic cells (Rafii et al, Gene Ther. 9:631-641 (2002)). For example, both putative EPCs with angioblastic potential and vessel wall-derived mature ECs may express similar endothelial-specific markers, including VEGFR2, Tie-1, Tie-2, VE-cadherin, CD34, and E-selectin. Similarly, markers, such as CD34, PECAM (CD31), Tie-1, Tie-2, von Willebrand factor (vWF), and VEGFR2 are expressed in both hematopietic cells and ECs (Rafii et al, Gene Ther. 9:631-641 (2002)). Furthermore, HSCs and even bone marrow-derived macrophages have been shown to transdifferentiate into endothelial-like cells (Schmeisser et al, Cardiovasc. Res. 49:671-680 (2001)). Recently, tissue-resident stem cells have been isolated from the heart, which are capable of differentiating into the endothelial lineage (Beltrami et al, Cell 114:763-776 (2003)). These data support the notion that it will be virtually impossible to identify the “true” EPCs based on the available markers. Conversely, highly purified EPCs may not be better suited for vascular repair, because several cell types (endothelial progenitors and supporting cells) may synergize in endothelialization and vascular healing. Hence, identification of the cell population that is enriched for EPCs and also contains necessary supporting cells is a critical step in enhancing therapeutic efficacy and reducing untoward side effects of cell therapeutic approaches. The present study provides evidence indicating that aging, in particular when it is accelerated by the presence of atherosclerosis risks, results in selective reduction/exhaustion of the supply of SLCs. Furthermore, lin⁻/cKit⁻/Sca-1⁻ cells constitute the bulk of the SLC population and are affected most profoundly by aging and hyperlipidemia. Remarkably, lin⁻/cKit⁻/Sca-1⁻ SLCs are more efficient in converting to a mature EC phenotype than other bone marrow fractions, including cEPCs and HSCs. These data indicate that lin⁻/cKit⁻/Sca-1⁻ SLCs may represent a cell population that is enriched for EPCs.

In addition to endothelial progenitors, which, for the most part, are presumably lineage restricted, two other stem/progenitors cell types—mesenchymal stem cells (MSCs) and multipotent adult progenitor cells (MAPCs)—with multipotent differentiation and extensive proliferation potential have been extensively investigated for vascular repair. MSCs are capable of stimulating angiogenesis and arteriogenesis after acute myocardial infarction (Silva et al, Circulation 111:150-156 (2005)). MAPCs copurify with MSCs and, when cultured with VEGF, differentiate into CD34⁺, VE-cadherin⁺, Flk1⁺ cells—a phenotype consistent with angioblasts—which subsequently differentiate into cells that express endothelial markers, functioning in vitro as mature endothelial cells and contributing to neoangiogenesis in vivo (Reyes et al, J. Clin. Invest. 109:337-346 (2002)). Since both cell types are lin−/cKit−/Sca-1, it is conceivable that the lin⁻/cKit⁻/Sca-1⁻ SLC population is enriched for MSCs and MAPCs. Indeed, the discovery of SLCs may provide an efficient alternative to MSCs and MAPCs, whose isolation requires extended culture and may introduce alterations to the cell phenotype.

In conclusion, the depletion of EPC-enriched lin⁻/cKit⁻/Sca-1⁻ SLCs in aging and atherosclerotic mice in combination with observations that decreased circulating EPC levels predict atherosclerosis disease outcome (Werner et al, N. Engl. J. Med. 353:999-1007 (2005)) and that the function of EPCs are impaired in high-risk patients (Hill et al, N. Engl. J. Med. 348:593-600 (2003)) suggest that the patients most in need of EPCs may be those who least possess them for autologous transplantation. Hence, approaches to enrich EPCs and/or enhance their function may be necessary to increase efficacy of bone marrow transplantation. Furthermore, lin⁻/cKit⁻/Sca-1⁻ SLCs may serve as a marker to screen candidate patients, in particular those with atherosclerosis, for their suitability for autologous bone marrow transplantation.

All documents and other information sources cited above are hereby incorporated in their entirety by reference.

What is claimed is:

1. An isolated population comprising lineage negative, cKit negative and Sca-1 negative (lin⁻/cKit⁻/Sca-1⁻) mammalian cells.

2. The population according to claim 1 wherein said cells are human cells.

3. The population according to claim 1 wherein said cells are isolated from bone marrow.

4. The population according to claim 1 wherein said cells are vascular endothelial progenitor cells.

5. The population according to claim 1 wherein said cells comprise a drug, a nucleic acid sequence encoding an anti-atherosclerotic product, or an image agent.

6. A method of treating atherosclerosis in a mammalian host comprising administering to said host lin⁻/cKit⁻/Sca-1⁻ cells in an amount sufficient to effect said treatment.

7. The method according to claim 6 wherein said lin⁻/cKit⁻/Sca-1⁻ cells are autologous cells.

8. The method according to claim 6 wherein said lin⁻/cKit⁻/Sca-1⁻ cells are heterologous cells.

9. The method according to claim 6 wherein said lin⁻/cKit⁻/Sca-1⁻ cells are expanded in vitro prior to administration.

10. The method according to claim 6 wherein said lin⁻/cKit⁻/Sca-1⁻ cells are administered via catheter or via injection.

11. The method according to claim 6 wherein said method is a prophylactic method.

12. The method according to claim 6 wherein said method is a therapeutic method.

13. The method according to claim 6 wherein said lin⁻/cKit⁻/Sca-1⁻ cells comprise a drug, a nucleic acid sequence encoding an anti-atherosclerotic product, or an imaging agent.

14. The method according to claim 13 wherein said product is protective against oxidative damage.

15. The method according to claim 14 wherein said product is superoxide dismutase or glutathione peroxidase.

16. The method according to claim 13 wherein said product is a component of the nitric oxide synthetic pathway, an agent that modulates Toll-like receptor activity, a serum cholesterol lowering polypeptide, or an agent that modulates expression or activity of fchd 531, fchd 540, fchd 545, or fchd 602 genes.

17. The method according to claim 13 wherein said product is glycogen synthase kinase 3β protein.

18. The method according to claim 6 wherein said mammalian host is a human.
